(19) United States
(12) Patent Application Publication
(10) Pub. No.: US 2026/0295177 A1
(43) Pub. Date: Oct. 1, 2026

(54) FLUID DELIVERY DEVICE FOR DETERMINING DEVICE IMPLANT LOCATION

(71) Applicant: Medtronic, Inc., Minneapolis, MN (US)

(72) Inventors: John D. Rogers, San Diego, CA (US); Todd M. Zielinski, McGrath, MN (US)

(21) Appl. No.: 19/479,035

(22) PCT Filed: Mar. 19, 2024
(86) PCT No.: PCT/US2024/020500

§ 371 Date: Oct. 27, 2025

Related U.S. Application Data
(60) Provisional application No. 63/498,719, filed on Apr. 27, 2023.

(51) Int. Cl. A61M 5/32

(52) U.S. Cl. CPC A61M 5/329; A61M 2205/13; A61M 2205/502

(57) ABSTRACT

A fluid delivery device includes a fluid reservoir configured to contain a fluid. A piercing member is in fluid communication with the fluid reservoir. A set of sensors is carried by the piercing member. The set of sensors is configured to: be positioned in the subcutaneous tissue when the piercing member has penetrated the subcutaneous tissue; and measure a signal from the subcutaneous tissue. Processing circuitry is configured to: determine a metric of the signal; determine whether the metric of the signal satisfies a condition; and based on whether the metric of the signal satisfies the condition, output a notification indicative of whether an implant location for an implantable medical device is satisfactory.

This application claims the benefit of U.S. Provisional Application Ser. No. 63/498,719, filed Apr. 27, 2023, the entire content of which is incorporated herein by reference.

### TECHNICAL FIELD

This disclosure is related to techniques for determining an implant location for an implantable medical device, such as a subcutaneous monitoring device.

### BACKGROUND

Some types of implantable medical device systems may be configured to monitor one or more physiological parameters of a patient. Such systems may include one or more sensors that detect signals associated with physiological parameters of a patient. These implantable medical devices (IMDs) may be implanted subcutaneously. IMDs may allow clinicians to obtain patient data without the patient being connected to an external machine and/or present in a clinic. An IMD that is configured to continuously record one or more physiological parameters allows clinicians to review data over a longer period of time as compared with systems that use external monitoring equipment in a simulated testing situation.

### SUMMARY

In general, body surface mapping techniques may be used to determine an implant location for an IMD. However, body surface mapping techniques may be inaccurate or imprecise (e.g., because signals sensed via cutaneous sensing methods may not represent subcutaneous tissue signal integrity). Accordingly, implanting an IMD at an implant location determined using body surface mapping techniques may result in sub-optimal diagnostic signal integrity (e.g., low signal strength).

In accordance with techniques of this disclosure, a fluid delivery device may be configured to deliver fluid (e.g., anesthetic fluid, saline, vasoconstrictors, etc.) to a patient (e.g., in preparation for implanting the IMD). During a local anesthetic injection procedure, the fluid delivery device may identify an implant location having desirable subcutaneous tissue signal integrity. Thus, techniques in accordance with this disclosure may advantageously replace body surface mapping procedures and other conventional techniques because subcutaneous signal measurement for determining implant location occurs during the administration of local anesthesia.

In some examples, a fluid delivery device comprises: a fluid reservoir configured to contain a fluid; a piercing member in fluid communication with the fluid reservoir, wherein the piercing member is configured to penetrate subcutaneous tissue of a patient; a plunger configured to eject the fluid from the fluid reservoir through a lumen defined by the piercing member; a set of sensors carried by the piercing member, wherein the set of sensors is configured to: be positioned in the subcutaneous tissue when the piercing member has penetrated the subcutaneous tissue; and measure a signal from the subcutaneous tissue; and processing circuitry configured to: determine a metric of the signal; determine whether the metric of the signal satisfies a condition; and based on whether the metric of the signal satisfies the condition, output a notification indicative of whether an implant location for an implantable medical device is satisfactory.

In some examples, a system comprises: a fluid delivery device comprising: a fluid reservoir configured to eject a fluid into a patient; a piercing member in fluid communication with the fluid reservoir, wherein the piercing member is configured to penetrate subcutaneous tissue of the patient; a plunger configured to eject the fluid from the fluid reservoir through a lumen defined by the piercing member; a set of sensors carried by the piercing member, wherein the set of sensors is configured to: be positioned in the subcutaneous tissue when the piercing member has penetrated the subcutaneous tissue; and measure a signal from the subcutaneous tissue; and communication circuitry; and an external device, wherein the communication circuitry is configured to communicate with the external device, and wherein the external device comprises processing circuitry configured to: determine a metric of the signal; determine whether the metric of the signal satisfies a condition; and based on whether the metric of the signal satisfies the condition, output a notification indicative of whether an implant location for an implantable medical device is satisfactory.

In some examples, a method comprises: positioning a set of sensors of a fluid delivery device in subcutaneous tissue of a patient, wherein the fluid delivery device comprises: a fluid reservoir configured to eject a fluid into the patient; a piercing member in fluid communication with the fluid reservoir, wherein the piercing member is configured to penetrate the subcutaneous tissue of the patient, and wherein the set of sensors is carried by the piercing member; a plunger configured to eject the fluid from the fluid reservoir through a lumen defined by the piercing member; and communication circuitry; measuring, by the set of sensors, a signal from the subcutaneous tissue; communicating, by the communication circuitry, the signal to an external device; determining, by processing circuitry of the external device, a metric of the signal; determining, by the processing circuitry, whether the metric of the signal satisfies a condition; and based on whether the metric of the signal satisfies the condition, outputting, by the processing circuitry, a notification indicative of whether an implant location for an implantable medical device is satisfactory.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating a fluid delivery device configured to sense a signal from subcutaneous tissue, in accordance with one or more techniques of this disclosure.

FIG. 2 is a block diagram of a fluid delivery device configured to sense a signal from subcutaneous tissue, in accordance with one or more techniques of this disclosure.

FIGS. 3A-3B are conceptual diagrams illustrating an example configuration of an IMD configured to be inserted under a patient's skin.

FIG. 4 is a conceptual diagram illustrating a fluid delivery device penetrating subcutaneous tissue of a patient, in accordance with one or more techniques of this disclosure.

FIG. 5 is a conceptual diagram illustrating a prospective implant location, in accordance with one or more techniques of this disclosure.

FIG. 6 is a conceptual diagram of a graphical user interface including data transmitted by a fluid delivery device, in accordance with one or more techniques of this disclosure.

FIG. 7 is a flow diagram illustrating an example method for using a fluid delivery device configured to sense a signal from subcutaneous tissue, in accordance with one or more techniques of this disclosure.

### DETAILED DESCRIPTION

As used herein, implantable medical device (IMD) refers to any medical device configured to be placed underneath the patient's skin. Examples an IMD include an insertable cardiac monitor (ICM), which is inserted underneath the skin using minimally invasive procedures, a medical diagnostic device (e.g., a miniaturized blood pressure monitor), a therapeutic device, etc. Other examples of IMDs include pacemakers and implantable cardioverter defibrillators (ICDs), which require more invasive surgical procedures to implant. In other words, the terms “implantable medical device” and “IMD” may refer to any device configured to be placed underneath the skin, regardless of the invasiveness of the procedures used to place the IMD underneath the skin.

IMDs, such as ICMs, may record one or more patient parameters using electrodes and other sensors. Clinicians may use the IMD to facilitate patient health monitoring and risk management. For example, the IMD can continuously (e.g., on a periodic or triggered basis without human intervention) sense one or more patient parameters while subcutaneously implanted in a patient over months or years and perform numerous operations per second to diagnose and/or treat a patient. As such, an IMD may be advantageous when a physician cannot be continuously present with the patient over weeks or months to evaluate the patient and/or where performing operations on patient parameter data (which may include weeks or months of data, if not more) could not practically be performed in the mind of a physician.

Inserting an IMD may include piercing the patient's skin, creating an opening underneath the skin, and placing the IMD within the opening at an implant location so that the IMD is configured to record patient data based on signals sensed from the location. In some examples, the IMD may be implanted via invasive surgical procedures that require the patient to be admitted to a hospital, undergo general anesthesia (local anesthesia is usually required for a surgical procedure involving an incision into the skin), and remain in the hospital to recover.

In general, body surface mapping techniques may be used to determine an implant location for the IMD. However, body surface mapping techniques may be inaccurate or imprecise (e.g., because signals sensed via cutaneous sensing methods may not represent subcutaneous tissue signal integrity). Accordingly, implanting an IMD at an implant location determined using body surface mapping techniques may result in sub-optimal diagnostic signal integrity (e.g., low signal strength). Diagnostic signal integrity is essential in order for the IMD to accurately diagnose a patient. Thus, any loss of fidelity in the diagnostic signal integrity may cause a corresponding increase in false-positive determinations.

In accordance with techniques of this disclosure, a fluid delivery device including a fluid reservoir (e.g., a syringe), a piercing member (e.g., a needle), and a plunger may be configured to subcutaneously measure physiological parameters of a patient. The fluid delivery device may include a set (e.g., one or more) of sensors positioned on the piercing member. In some examples, the sensor selection (e.g., type of sensors on the piercing member) and sensor configuration (e.g., spacing of sensors on the piercing member) may be specific to the IMD to be implanted in a patient in order to increase the likelihood that subcutaneous measurements collected by the set of sensors on the piercing member are representative of the diagnostic signal integrity for the IMD.

The fluid delivery device may be configured to deliver fluid to a patient (e.g., in preparation for implanting the IMD). For example, during a local anesthetic injection procedure, the fluid delivery device may identify (e.g., based on subcutaneous measurements collected by the set of sensors on the piercing member) an implant location (e.g., implant position, implant orientation, etc.) having desirable subcutaneous tissue signal integrity. Thus, techniques in accordance with this disclosure may advantageously replace body surface mapping procedures and other conventional techniques because subcutaneous signal measurement for determining implant location occurs during the administration of local anesthesia.

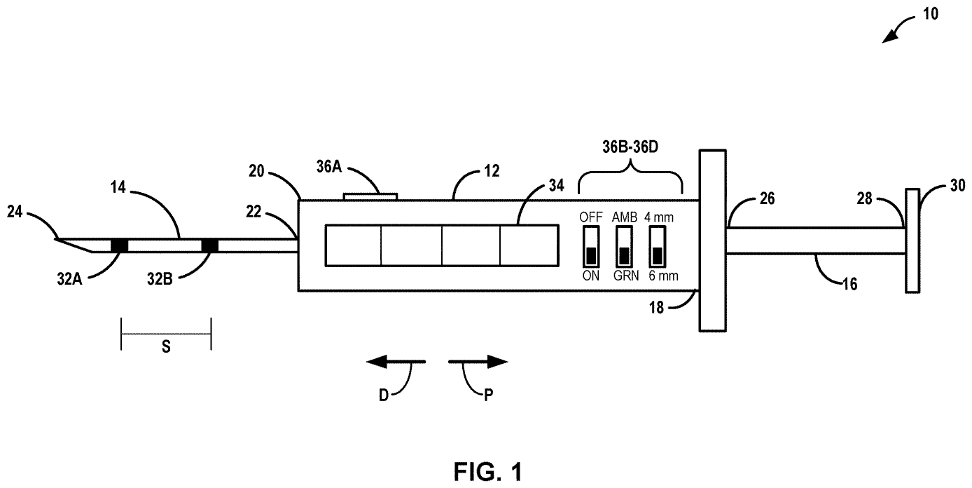

FIG. 1 is a conceptual diagram illustrating a fluid delivery device 10 in accordance with one or more techniques of this disclosure. Fluid delivery device 10 may include a fluid reservoir 12 (e.g., a syringe), a piercing member 14 (e.g., a needle), and a plunger 16. Fluid reservoir 12 may include a proximal end 18 (“fluid reservoir proximal end 18”) and a distal end 20 (“fluid reservoir distal end 20”). Piercing member 14 may include a proximal end 22 (“piercing member proximal end 22”) and a piercing member distal end 24 (“piercing member distal end 24”). Plunger 16 may include a proximal end 26 (“plunger proximal end 26”) and a distal end 28 (“plunger distal end 28”). Fluid reservoir 12 may be configured to contain fluid (e.g., anesthetic fluid, saline, vasoconstrictors, etc.), and fluid reservoir 12 and piercing member 14 may be in fluid communication with each other. Fluid reservoir 12 and plunger 16 may be mechanically coupled, and plunger 16 may be configured to translate within fluid reservoir 12.

Fluid delivery device 10 may be configured to deliver fluid contained within fluid reservoir 12 to a target area within a patient via piercing member 14. For example, piercing member 14 may be configured to pierce or otherwise penetrate subcutaneous tissue of the patient to create an opening. Plunger 16 may be configured to eject fluid from reservoir 12 through a lumen defined by piercing member 14 by, for example, translating in the distal direction (indicated by the arrow “D”) within fluid reservoir 12. A force exerted (e.g., by a clinician) in a distal direction on a handle 30 of fluid delivery device 10 may cause plunger 16 to translate in a distal direction within fluid reservoir 12.

Fluid delivery device 10 may include or otherwise carry a set (e.g., one or more) of sensors 32A-32N (collectively, “sensors 32”). Sensors 32 may include sensors 32A-32B which are shown and labeled in FIG. 1. However, for ease of illustration, not all of sensors 32 are shown and labeled in FIG. 1. Sensors 32 may be positioned on a surface of piercing member 14 (e.g., an exterior surface of piercing member 14, an interior surface defining the lumen of piercing member 14, etc.). While only two sensors are shown in FIG. 1 for ease of illustration, any number of sensors may be positioned on piercing member 14. Sensors 32 may be positioned on piercing member 14 such that when piercing member 14 has penetrated the subcutaneous tissue (per an anesthetic injection procedure), sensors 32 may be positioned in the subcutaneous tissue. Examples of sensors 32 may include an electrode array (e.g., one or more electrodes), a photoemitter, a photodetector, an accelerometer, an inductive sensor, an electromagnetic sensor, an impedance sensor, etc.

Sensors 32 may be configured to measure a signal from the subcutaneous tissue. For example, sensor 32 may be configured to subcutaneously measure physiological parameters of a patient. Examples of physiological parameters may include electrocardiograms (ECG), arterial pulse pressure waveforms, autonomic nervous system activity, waveform amplitudes, area under the curve, timing intervals, etc. With respect to determining implant locations for IMDs, subcutaneously measuring physiological parameters may be superior to cutaneous sensing methods (e.g., body surface mapping techniques) because signals sensed via subcutaneous sensing methods may more accurately represent subcutaneous tissue signal integrity. For example, when sensors 32 are positioned at or proximate a prospective implant site, the measurements by sensors 32 may be substantially similar (e.g., in terms of signal strength, signal quality, etc.) to measurements by sensors of an IMD positioned at the prospective implant site.

In some examples, the selection and configuration of sensors 32 may match or otherwise correspond to the selection and configuration of sensors carried by an IMD to increase the likelihood that subcutaneous measurements collected by sensors 32 are representative of the diagnostic signal integrity for the IMD. For example, if the IMD to be implanted includes an electrode array spaced about 4 centimeters (cm) apart, sensors 32 may likewise include an electrode array space about 4 cm apart on piercing member 14. Thus, the position of sensors carried by piercing member 14 may match or otherwise correspond to the position of sensors carried by the IMD. For example, the spacing of sensors carried by piercing member 14 may match or otherwise correspond to the spacing of sensors carried by the IMD.

Fluid delivery device 10 may determine a metric of the signal measured by sensors 32 from the subcutaneous tissue. The metric of the signal may represent a measure of the subcutaneous tissue signal integrity. For example, fluid delivery device 10 may determine a signal strength (e.g., an amplitude, such as a peak-to-peak amplitude or mean rectified amplitude) of the signal measured by sensors 32. Measuring signal strength may be advantageous because an adequate signal strength (e.g., a signal strength that equals or exceeds a threshold signal strength) may be necessary for an IMD to accurately process the signal for diagnostic purposes. For example, if the signal strength from the subcutaneous tissue is too weak and therefore inadequate, the IMD may not be able to consistently sense a signal, in turn preventing diagnosis based on the signal.

Additionally or alternatively, fluid delivery device 10 may determine a signal quality (e.g., a noisiness) of the signal measured by sensors 32. Measuring signal quality may be advantageous because an adequate signal quality (e.g., a signal quality that equals or exceeds a threshold signal quality) may be necessary for an IMD to accurately process the signal for diagnostic purposes. For example, if the signal quality from the subcutaneous tissue is too low and therefore inadequate, processing of the signal by the IMD may result in an undesirably high false-positive rate because of the noise in the signal. In some examples, reservoir 12 may include a display 34 configured to display the metric of the signal (e.g., as a bar graph display).

Processing circuitry of fluid delivery device 10 may filter the physiologic signal measured by fluid delivery device 10 (e.g., ECG, arterial pulse pressure waveform, heart sounds, etc.) based on a desired bandwidth of the signal content. In some examples, the processing circuitry may assess Signal to Noise Ratios, waveform template matching, waveform fiducial points, timing intervals, area under the curve, etc., to determine if the signal quality is adequate to determine optimal device implant location.

In any case, fluid delivery device 10 may determine whether the metric of the signal satisfies a condition. For example, fluid delivery device 10 may determine that the metric satisfies the condition if the metric is equal to or greater than a threshold value. Based on whether the metric of the signal satisfies the condition, fluid delivery device 10 may output a notification indicative of whether an implant location (e.g., the subcutaneous tissue penetrated by piercing member 14 and sensed by sensors 34) for an IMD is satisfactory. In some examples, display 34 may display the notification indicating whether the implant location is satisfactory. For example, display 34 may display colors, shapes, and/or provide some other form of visual information.

In some examples, fluid delivery device 10 may include one or more input devices 36A-36N (collectively, “input devices 36”). Input devices 36 may include input devices 36A-36D which are shown and labeled in FIG. 1. However, for ease of illustration, not all of input devices 36 are shown and labeled in FIG. 1. A user of fluid delivery device 10 may use input devices 36 to operate and configure sensors 32. For example, the user may use input device 36A to cause sensors 32 to sense a signal, input device 36B to turn sensors 32 on and off, input device 36C to change the color of light emitted by a photoemitter positioned on piercing member 14 between amber and green, input device 36D to select a spacing (represented by ‘S’ in FIG. 1) between active sensors 32, e.g., between a photodetector positioned on piercing member 14 spaced 4 millimeters (mm) from the photoemitter and a photodetector positioned on piercing member 14 spaced 6 mm, etc. In some examples, reservoir 12 may include one or more of input devices 36.

While FIG. 1 illustrates four input devices, it should be understood that fluid delivery device 10 may include more or fewer input devices for configuring various aspects of sensors 32. For example, fluid delivery device 10 may include an input device for selecting between various sensors, such as an electrode array, a photoemitter, a photodetector, an accelerometer, an inductive sensor, an electromagnetic sensor, an impedance sensor, etc. In another example, fluid delivery device 10 may include an input device for selecting active electrodes of an electrode array positioned on piercing member 14 that are spaced 4 cm from each other and active electrodes that are spaced 6 cm from each other. The configurations of sensors 32 associated with input devices 36 described herein are examples provided for the purpose of explanation, and other configurations of sensors 32 associated with input devices 36 (such as configurations relating to selection of the sensors, the spacing of the sensors, etc.) are contemplated by this disclosure.

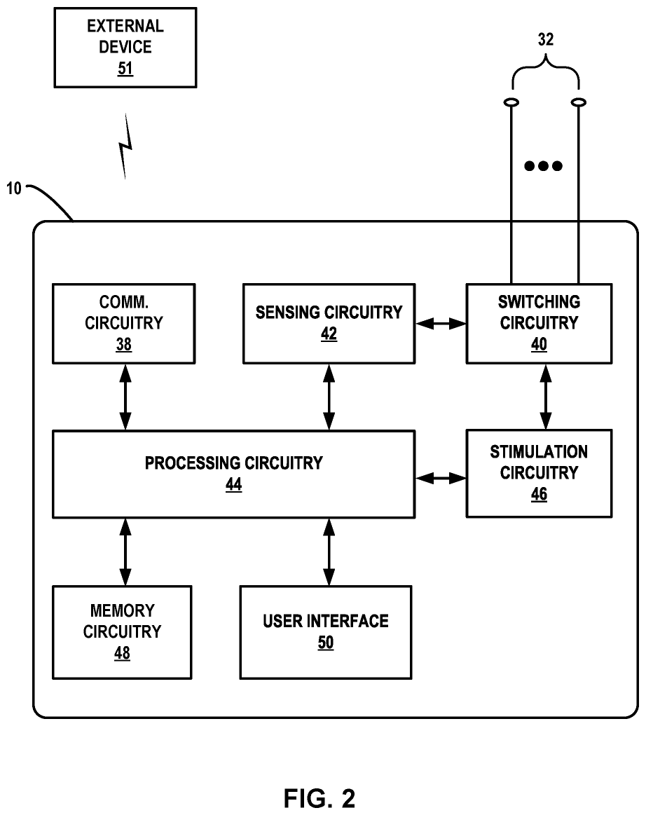

FIG. 2 is a block diagram illustrating a configuration of fluid delivery device 10 in accordance with techniques of this disclosure. As shown in FIG. 2, fluid delivery device 10 includes communication circuitry 38 (“COMM circuitry 38”), switching circuitry 40, sensing circuitry 42, lircuitry 44, stimulation circuitry 46, memory circuitry 48, and a user interface 50. User interface 50 may include display 34 and input devices 36. Fluid delivery device 10 may be electrically connected to sensors 32. In some examples, fluid delivery device 10 may be electrically connected to sensors 32 via piercing member 14.

Processing circuitry 44 may include fixed function circuitry and/or programmable processing circuitry. Processing circuitry 44 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 44 may include multiple components, such as any combination of one or more microprocessors, one or more controllers, one or more DSPs, one or more ASICs, or one or more FPGAs, as well as other discrete or integrated logic circuitry. The functions attributed to processing circuitry 44 herein may be embodied as software, firmware, hardware or any combination thereof.

Stimulation circuitry 46 may be configured to generate and deliver a type of therapy, such as electrical therapy. Stimulation circuitry 46 may be selectively coupled to sensors 32 via switching circuitry 40 as controlled by processing circuitry 44 to, for example, deliver a set of stimulation pulses to tissue of the patient.

Sensing circuitry 42 may be selectively coupled to sensors 32 via switching circuitry 40 as controlled by processing circuitry 44 to, for example, sense electrical signals from tissue of a patient. In some examples, sensing circuitry 42 may include one or more filters and amplifiers for filtering and amplifying signals received from electrodes 32. Sensing circuitry 42 may include analog-to-digital conversion circuitry for converting the signals to digital samples for analysis by processing circuitry 44 and/or storage in memory circuitry 48.

COMM circuitry 38 may include any suitable hardware, firmware, software or any combination thereof configured to communicate with another device, such as an external device 51. Under the control of processing circuitry 44, COMM circuitry 38 may receive downlink telemetry from, as well as send uplink telemetry to external device 51 or another device with the aid of an internal or external antenna. COMM circuitry 38 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

External device 51 may be a computing device, e.g., used in a home, ambulatory, clinic, or hospital setting, to communicate with fluid delivery device 10 via wireless telemetry. External device 51 may be coupled to a remote patient monitoring system, such as Carelink®. External device 51 may be, for example, a programmer, an external monitor, a mobile computing device (e.g., a tablet computer or a smart phone), etc. In some examples, external device 51 may be used to program commands or operating parameters into fluid delivery device 10 for controlling its functioning, such as configuring sensors 32. External device 51 may be used to interrogate fluid delivery device 10 to retrieve data, such as the subcutaneous tissue signal and associated metrics. The interrogation may be automatic (e.g., according to a schedule) or in response to a remote or local user command, e.g., initiated via user interface 50 or a user interface of external device 51.

In some examples, memory circuitry 48 includes computer-readable instructions that, when executed by processing circuitry 44, cause processing circuitry 44, and in turn fluid delivery device 10, to perform various functions attributed to fluid delivery device 10 herein. Memory circuitry 48 may include any volatile, non-volatile, magnetic, optical, or electrical media, such as a random-access memory (RAM), read-only memory (ROM), non-volatile RAM (NVRAM), electrically-erasable programmable ROM (EEPROM), ferroelectric RAM (FRAM), dynamic random-access memory (DRAM), flash memory, or any other digital media. Memory circuitry 48 may store, as examples, programmed values for one or more operational parameters of processing circuitry 44. Memory circuitry 48 may also store data collected by fluid delivery device 10 for transmission to another device using COMM circuitry 38 and/or further analysis by processing circuitry 44.

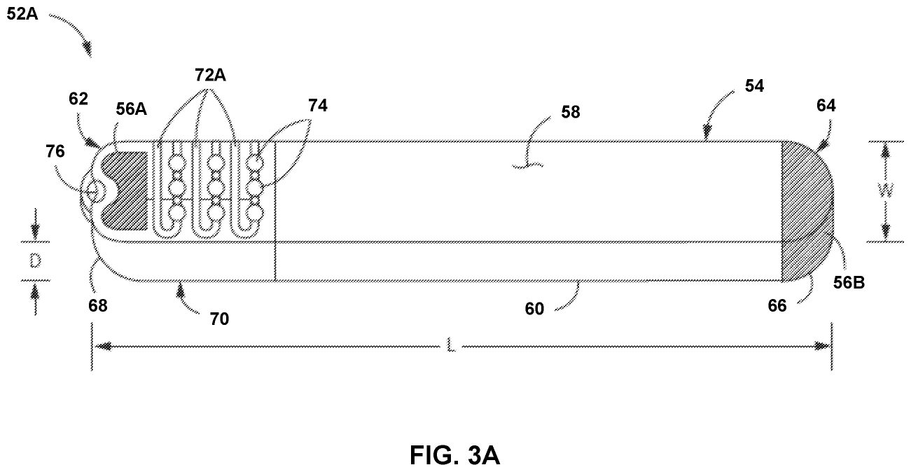

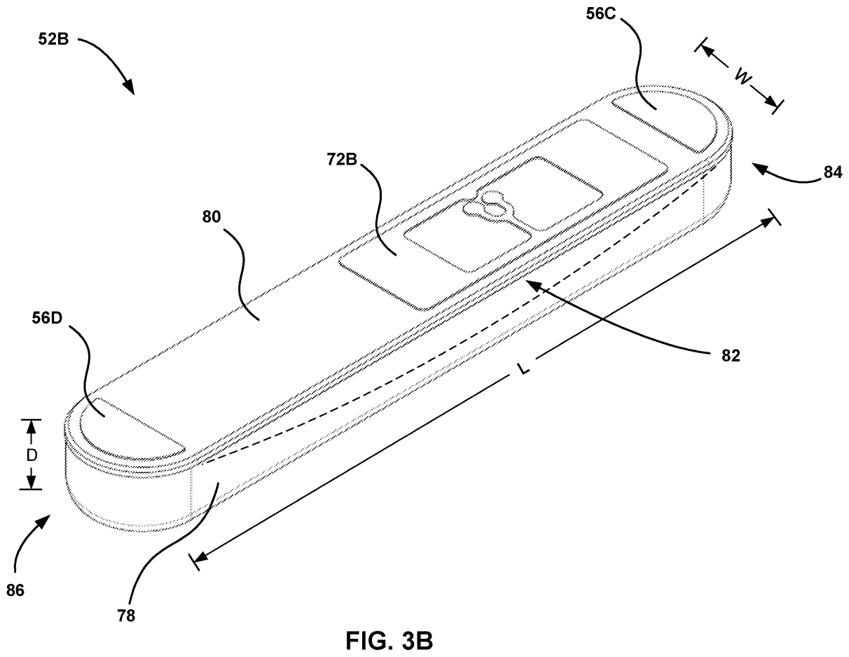

FIG. 3A is a perspective drawing illustrating an IMD 52, which may be an example configuration of the IMD described with respect to FIG. 1. In the example shown in FIG. 3A, IMD 52A may be embodied as a monitoring device having housing 54, proximal electrode 56A and distal electrode 56B. Housing 54 may further comprise first major surface 58, second major surface 60, proximal end 62, and distal end 64. Housing 54 encloses electronic circuitry located inside the IMD 52A and protects the circuitry contained therein from body fluids. Housing 54 may be hermetically sealed and configured for subcutaneous implantation. Electrical feedthroughs provide electrical connection of electrodes 16A and 16B.

In the example shown in FIG. 3A, IMD 52A is defined by a length L, a width W and thickness or depth D and is in the form of an elongated rectangular prism wherein the length L is much larger than the width W, which in turn is larger than the depth D. In one example, the geometry of the IMD 52A, in particular a width W greater than the depth D, is selected to allow IMD 52A to be inserted under the skin of the patient using a minimally invasive procedure and to remain in the desired orientation during insertion. For example, the device shown in FIG. 3A includes radial asymmetries (notably, the rectangular shape) along the longitudinal axis that maintains the device in the proper orientation following insertion. For example, the spacing between proximal electrode 56A and distal electrode 56B may range from 5 millimeters (mm) to 55 mm, 30 mm to 55 mm, 35 mm to 55 mm, and from 40 mm to 55 mm and may be any range or individual spacing from 5 mm to 60 mm.

IMD 52A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 58 may range from 3 mm to 15, mm, from 3 mm to 10 mm, or from 5 mm to 15 mm, and may be any single or range of widths between 3 mm and 15 mm. The thickness of depth D of IMD 52A may range from 2 mm to 15 mm, from 2 mm to 9 mm, from 2 mm to 5 mm, from 5 mm to 15 mm, and may be any single or range of depths between 2 mm and 15 mm. In addition, IMD 52A according to an example of the present disclosure is has a geometry and size designed for ease of implant and patient comfort. Examples of IMD 52A may have a volume of three cubic centimeters (cm) or less, 1.5 cubic cm or less or any volume between three and 1.5 cubic centimeters.

In the example shown in FIG. 3A, once inserted within the patient, the first major surface 58 faces outward, toward the skin of the patient while the second major surface 60 is located opposite the first major surface 58. In addition, in the example shown in FIG. 3A, proximal end 62 and distal end 64 are rounded to reduce discomfort and irritation to surrounding tissue once inserted under the skin of the patient.

Proximal electrode 56A is at or proximate to proximal end 62, and distal electrode 56B is at or proximate to distal end 64. Proximal electrode 56A and distal electrode 56B are used to sense cardiac EGM signals, e.g., ECG signals, thoracically outside the ribcage, which may be sub-muscularly or subcutaneously. EGM signals may be stored in a memory of IMD 52A, and data may be transmitted via integrated antenna 72A to another device, which may be another implantable device or an external device, such as external device 51. In some example, electrodes 56A and 56B may additionally or alternatively be used for sensing any bio-potential signal of interest, which may be, for example, an EGM, EEG, EMG, or a nerve signal, or for measuring impedance, from any implanted location.

In the example shown in FIG. 3A, proximal electrode 56A is at or in close proximity to the proximal end 62 and distal electrode 56B is at or in close proximity to distal end 64. In this example, distal electrode 56B is not limited to a flattened, outward facing surface, but may extend from first major surface 58 around rounded edges 66 and/or end surface 68 and onto the second major surface 60 so that the electrode 56B has a three-dimensional curved configuration. In some examples, electrode 56B is an uninsulated portion of a metallic, e.g., titanium, part of housing 54.

In the example shown in FIG. 3A, proximal electrode 56A is located on first major surface 58 and is substantially flat, and outward facing. However, in other examples proximal electrode 56A may utilize the three dimensional curved configuration of distal electrode 56B, providing a three dimensional proximal electrode (not shown in this example). Similarly, in other examples distal electrode 56B may utilize a substantially flat, outward facing electrode located on first major surface 58 similar to that shown with respect to proximal electrode 56A.

The various electrode configurations allow for configurations in which proximal electrode 56A and distal electrode 56B are located on both first major surface 58 and second major surface 60. In other configurations, such as that shown in FIG. 3A, only one of proximal electrode 56A and distal electrode 56B is located on both major surfaces 58 and 60, and in still other configurations both proximal electrode 56A and distal electrode 56B are located on one of the first major surface 58 or the second major surface 60 (e.g., proximal electrode 56A located on first major surface 58 while distal electrode 56B is located on second major surface 60). In another example, IMD 52A may include electrodes on both major surfaces 58 and 60 at or near proximal end 62 and distal end 64 of IMD 52A, such that a total of four electrodes are included on IMD 52A. Electrodes 56A and 56B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 3A, proximal end 62 includes a header assembly 70 that includes one or more of proximal electrode 56A, integrated antenna 72A, anti-migration projections 74, and/or suture hole 76. Integrated antenna 72A is located on the same major surface (i.e., first major surface 58) as proximal electrode 56A and is also included as part of header assembly 70. Integrated antenna 72A allows IMD 52A to transmit and/or receive data. In other examples, integrated antenna 72A may be formed on the opposite major surface as proximal electrode 56A, or may be incorporated within the housing 54 of IMD 52A. In the example shown in FIG. 3A, anti-migration projections 74 are located adjacent to integrated antenna 72A and protrude away from first major surface 58 to prevent longitudinal movement of the device. In the example shown in FIG. 3A, anti-migration projections 74 include a plurality (e.g., nine) small bumps or protrusions extending away from first major surface 58. As discussed above, in other examples anti-migration projections 74 may be located on the opposite major surface as proximal electrode 56A and/or integrated antenna 72A. In addition, in the example shown in FIG. 3A, header assembly 70 includes suture hole 76, which provides another means of securing IMD 52A to the patient to prevent movement following insertion. In the example shown, suture hole 76 is located adjacent to proximal electrode 56A. In one example, header assembly 70 is a molded header assembly made from a polymeric or plastic material, which may be integrated or separable from the main portion of IMD 52A.

As described above, the selection and configuration of sensors 32 may match or otherwise correspond to the selection and configuration of sensors carried by IMD 52A to increase the likelihood that subcutaneous measurements collected by sensors 32 are representative of the diagnostic signal integrity for IMD 52A. For example, sensors 32 may include an electrode array configured in a similar manner as proximal electrode 56A and distal electrode 56B. Additionally, the positioning of sensors 32 may match the spacing between proximal electrode 56A and distal electrode 56B (and/or other sensors carried by IMD 52A). For example, the spacing between electrodes of the electrode array of sensors 32 may range from 5 millimeters (mm) to 55 mm, 30 mm to 55 mm, 35 mm to 55 mm, and from 40 mm to 55 mm and may be any range or individual spacing from 5 mm to 60 mm.

FIG. 3B is a perspective drawing illustrating another IMD 52B, which may be another example configuration of IMD 52 from FIG. 1 as an ICM configured to record one or more patient parameters. IMD 52B of FIG. 3B may be configured substantially similarly to IMD 52A of FIG. 3A, with differences between them discussed herein.

IMD 52B may include a leadless, subcutaneously-implantable monitoring device, e.g. an ICM. IMD 52B includes housing having a base 78 and an insulative cover 80. A proximal electrode 56C and a distal electrode 56D may be formed or placed on an outer surface of cover 80. Various circuitries and components of IMD 52B, e.g., described below with respect to FIG. 3, may be formed or placed on an inner surface of cover 80, or within base 78. In some examples, a battery or other power source of IMD 52B may be included within base 78. In the illustrated example, antenna 72B is formed or placed on the outer surface of cover 80, but may be formed or placed on the inner surface in some examples. In some examples, insulative cover 80 may be positioned over an open base 78 such that base 78 and cover 80 enclose the circuitries and other components and protect them from fluids such as body fluids. The housing including base 70 and insulative cover 72 may be hermetically sealed and configured for subcutaneous implantation.

Circuitries and components may be formed on the inner side of insulative cover 80, such as by using flip-chip technology. Insulative cover 80 may be flipped onto a base 78. When flipped and placed onto base 78, the components of IMD 52B formed on the inner side of insulative cover 80 may be positioned in a gap 82 defined by base 78. Proximal electrode 56C and distal electrode 56D and antenna 72B may be electrically connected to circuitry formed on the inner side of insulative cover 80 through one or more vias (not shown) formed through insulative cover 80. Insulative cover 80 may be formed of sapphire (i.e., corundum), glass, parylene, and/or any other suitable insulating material. Base 78 may be formed from titanium or any other suitable material (e.g., a biocompatible material Proximal electrode 56C and distal electrode 56D may be formed from any of stainless steel, titanium, platinum, iridium, or alloys thereof. In addition, proximal electrode 56C and distal electrode 56D may be coated with a material such as titanium nitride or fractal titanium nitride, although other suitable materials and coatings for such electrodes may be used.

In the example shown in FIG. 3B, the housing of IMD 52B defines a length L, a width W and thickness or depth D and is in the form of an elongated rectangular prism wherein the length L is much larger than the width W, which in turn is larger than the depth D, similar to IMD 52A of FIG. 3A. For example, the spacing between proximal electrode 56C and distal electrode 56D may range from 5 mm to 50 mm, from 30 mm to 50 mm, from 35 mm to 45 mm, and may be any single spacing or range of spacings from 5 mm to 50 mm, such as approximately 40 mm. In addition, IMD 52B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 52B may range from 2 mm to 15 mm, from 5 mm to 15 mm, or from 3 mm to 5 mm, and may be any single depth or range of depths between 2 mm and 15 mm, such as approximately 4 mm. IMD 52B may have a volume of three cubic centimeters (cm) or less, or 1.5 cubic cm or less, such as approximately 1.4 cubic cm.

In the example shown in FIG. 3B, once inserted subcutaneously within the patient, outer surface of cover 80 faces outward, toward the skin of the patient. In addition, as shown in FIG. 3B, proximal end 84 and distal end 86 are rounded to reduce discomfort and irritation to surrounding tissue once inserted under the skin of the patient. In addition, edges of IMD 52B may be rounded.

As described above, the selection and configuration of sensors 32 may match or otherwise correspond to the selection and configuration of sensors carried by IMD 52B to increase the likelihood that subcutaneous measurements collected by sensors 32 are representative of the diagnostic signal integrity for IMD 52B. For example, sensors 32 may include an electrode array configured in a similar manner as proximal electrode 56C and distal electrode 56DB. Additionally, the positioning of sensors 32 may match the spacing between proximal electrode 56C and distal electrode 56D (and/or other sensors carried by IMD 52B). For example, the spacing between electrodes of the electrode array of sensors 32 may range from 5 mm to 50 mm, from 30 mm to 50 mm, from 35 mm to 45 mm, and may be any single spacing or range of spacings from 5 mm to 50 mm, such as approximately 40 mm.

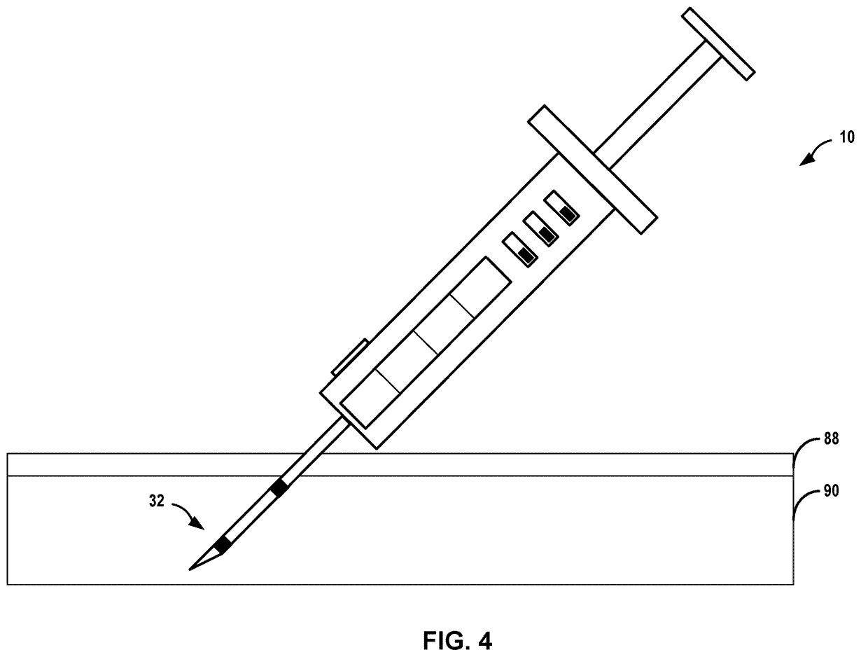

FIG. 4 is a conceptual diagram illustrating fluid delivery device 10 with piercing member 14 penetrating cutaneous tissue 88 and subcutaneous tissue 90 of a patient. Fluid delivery device 10 may penetrate cutaneous tissue 88 and subcutaneous tissue 90 per an anesthetic injection procedure. As shown in FIG. 4, when piercing member 14 has penetrated subcutaneous tissue 90, sensors 32 may be positioned in subcutaneous tissue 90 such that sensors 32 may sense signals from subcutaneous tissue 90.

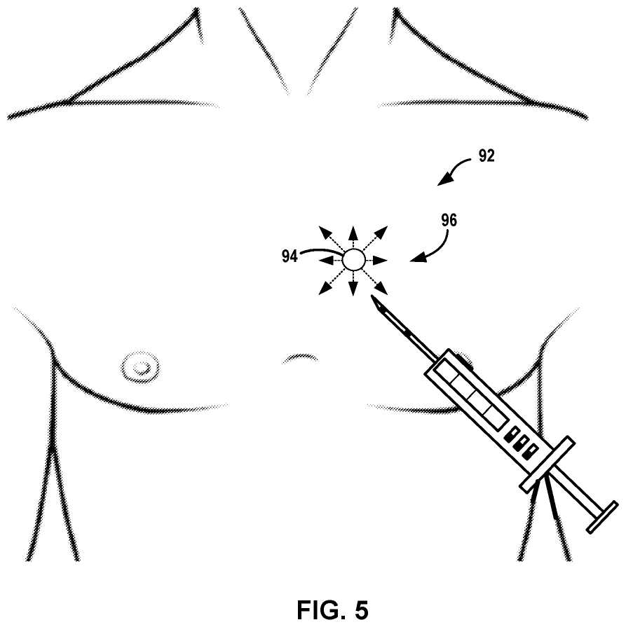

FIG. 5 is a conceptual diagram illustrating a prospective implant location 92 (“implant location 92”) for subcutaneously positioning an IMD (e.g., IMD 52A or IMD 52B). Implant location 92 may include an implant position 94 and an implant orientation 96. As used herein, implant position may refer to the position of the centroid of the IMD relative to the body of the patient, and implant orientation may refer to the angle of the IMD relative to the body of the patient. Implant position 94 and implant orientation 96 may each affect diagnostic signal integrity. That is, diagnostic signal integrity may vary based on implant position 94 and implant orientation of the IMD.

Fluid delivery device 10 may be configured to indicate whether implant position 94 and implant orientation 96 are suitable for the IMD. For example, piercing member 14 may penetrate the subcutaneous tissue at implant position 94 at an angle of entry corresponding to, for example, one of the dashed arrows shown in FIG. 5. In this way, sensors 32 may be positioned at implant position 94 and at an angle matching a possible orientation of sensors carried by the IMD. Thus, the signal measured by sensors 32 from the subcutaneous tissue may correspond to a signal that would be measured by sensors carried by the IMD if the IMD was implanted at implant position 94 with implant orientation 96.

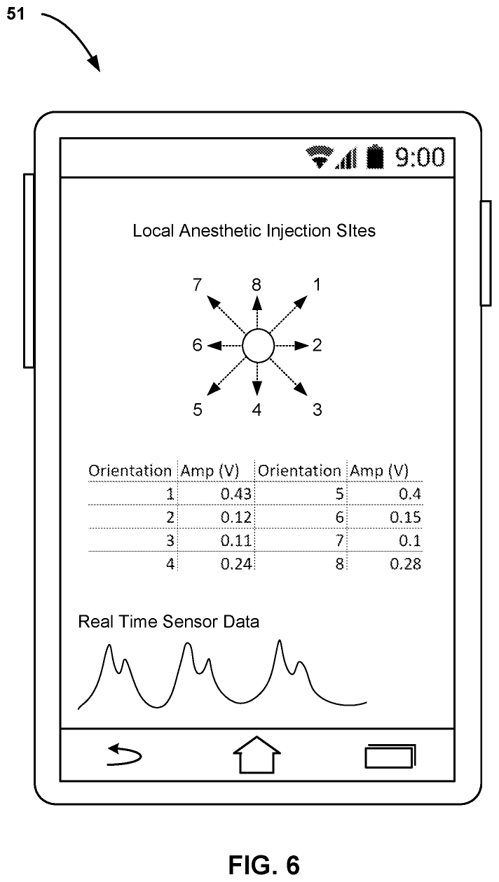

FIG. 6 is a conceptual diagram illustrating a graphical user interface (GUI) displayed by external device 51. As described above, fluid delivery device 10 may be configured to communicate (e.g., communication circuitry 38) with external device 51. In some examples, fluid delivery device 10 may transmit the signal measured by sensors 32 and a metric of the signal (e.g., a voltage amplitude of the signal). External device 51 may display the signal measured by sensors 32 (e.g., in real-time) and the metric of the signal for each implant orientation. In some examples, responsive to the metric of the signal satisfying a condition (e.g., the metric is equal to or greater than a threshold, such as a voltage amplitude of 0.25), external device 51 may output a notification indicative of whether the implant location is satisfactory.

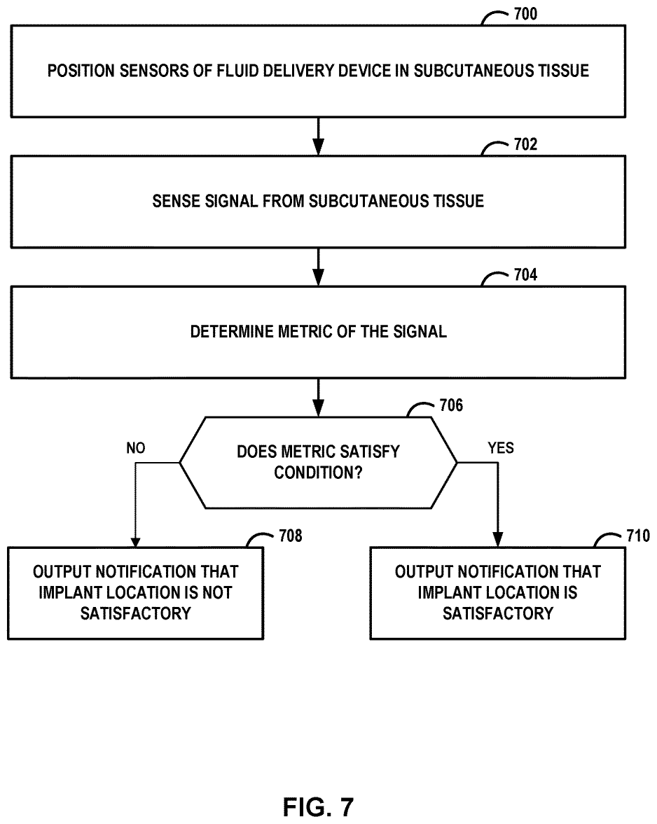

FIG. 7 is a flow diagram illustrating an example method for using fluid delivery device 10. Fluid delivery device 10 may pierce the skin of a patient using piercing member 14 to create an opening. Piercing member 14 may pierce the skin at implant location 92, where implant location 92 includes implant position 94 and implant orientation 96. When piercing member 14 has penetrated subcutaneous tissue, sensors 32 may be positioned in the subcutaneous tissue (700).

Sensors 32 may sense a signal from the subcutaneous tissue (702). The operation and configuration of sensors 32 may be in accordance with inputs from input devices 36. For example, sensors 32 may sense the signal in response to actuation of input device 36A. The selection and configuration of sensors 32 may match or otherwise correspond to the selection and configuration of the IMD to be implanted in the patient at implant location 92.

Fluid delivery device 10 may determine a metric of the signal measured by sensors 32 from the subcutaneous tissue (704). The metric of the signal may represent a measure of the subcutaneous tissue signal integrity. For example, fluid delivery device 10 may determine a signal strength of the signal measured by sensors 32. Additionally or alternatively, fluid delivery device 10 may determine a signal quality of the signal measured by sensors 32.

Processing circuitry (e.g., processing circuitry 44, processing circuitry in external device 51, other processing circuitry, etc.) may determine whether the metric of the signal satisfies a condition (706). For example, the processing circuitry may determine whether the signal strength is equal to or greater than a threshold value. In another example, the processing circuitry may determine whether the signal quality is equal to or greater than a threshold value. Other examples are contemplated by this disclosure, such as examples where the processing circuitry may determine whether the metric of the signal is equal to or less than a threshold value.

In any case, responsive to the processing circuitry determining that the metric of the signal does not satisfy the condition (“NO” branch of 706), the processing circuitry may output a notification that the implant location is not satisfactory (708). For example, fluid delivery device 10, external device 51, etc., may display visual information indicating that the implant location is not satisfactory. In turn, another implant location may be tested.

Responsive to the processing circuitry determining that the metric of the signal does not satisfy the condition (“YES” branch of 706), the processing circuitry may output a notification that the implant location is satisfactory. For example, fluid delivery device 10, external device 51, etc., may display visual information indicating that the implant location is satisfactory. IMD may then be implanted at the implant location.

Advantages of the techniques of this disclosure may include improving the diagnostic accuracy of an IMD in a manner that is relatively noninvasive. For example, testing the diagnostic signal integrity using fluid delivery device 10 may ensure that the signal from the subcutaneous tissue is adequate for the IMD to render an accurate diagnosis (e.g., per the garbage in, garbage out concept). Additionally, the testing may be done at about the same time as the injection of anesthetic, which is already generally part of a surgical procedure. Thus, the techniques may be convenient, quick, and effective, improving patient outcomes.

This disclosure includes various examples, such as the following examples.
Example 1: A fluid delivery device includes a fluid reservoir configured to contain a fluid; a piercing member in fluid communication with the fluid reservoir, wherein the piercing member is configured to penetrate subcutaneous tissue of a patient; a plunger configured to eject the fluid from the fluid reservoir through a lumen defined by the piercing member; a set of sensors carried by the piercing member, wherein the set of sensors is configured to: be positioned in the subcutaneous tissue when the piercing member has penetrated the subcutaneous tissue; and measure a signal from the subcutaneous tissue; and processing circuitry configured to: determine a metric of the signal; determine whether the metric of the signal satisfies a condition; and based on whether the metric of the signal satisfies the condition, output a notification indicative of whether an implant location for an implantable medical device is satisfactory.
Example 2: The fluid delivery device of example 1, wherein the implant location includes implant position and implant orientation.
Example 3: The fluid delivery device of example 1 or 2, wherein a spacing of the set of sensors carried by the piercing member corresponds to a spacing of a set of sensors carried by the implantable medical device.
Example 4: The fluid delivery device of any of examples 1 to 3, wherein the metric of the signal indicates one or more of a signal strength or a signal quality.
Example 5: The fluid delivery device of any of examples 1 to 4, wherein the fluid reservoir includes a display configured to display the metric of the signal.
Example 6: The fluid delivery device of any of examples 1 to 5, wherein the fluid includes at least one of anesthetic fluid, saline, or vasoconstrictors.
Example 7: The fluid delivery device of any of examples 1 to 6, wherein the set of sensors include at least one of an electrode array, a photoemitter, a photodetector, an accelerometer, an inductive sensor, an electromagnetic sensor, or an impedance sensor.
Example 8: The fluid delivery device of any of examples 1 to 7, wherein the fluid reservoir includes a set of input devices, and wherein the processing circuitry is configured to configure the set of sensors based on an input from the one or more input devices.
Example 9: A system includes a fluid delivery device includes a fluid reservoir configured to eject a fluid into a patient; a piercing member in fluid communication with the fluid reservoir, wherein the piercing member is configured to penetrate subcutaneous tissue of the patient; a plunger configured to eject the fluid from the fluid reservoir through a lumen defined by the piercing member; a set of sensors carried by the piercing member, wherein the set of sensors is configured to: be positioned in the subcutaneous tissue when the piercing member has penetrated the subcutaneous tissue; and measure a signal from the subcutaneous tissue; and communication circuitry; and an external device, wherein the communication circuitry is configured to communicate with the external device, and wherein the external device includes processing circuitry configured to: determine a metric of the signal; determine whether the metric of the signal satisfies a condition; and based on whether the metric of the signal satisfies the condition, output a notification indicative of whether an implant location for an implantable medical device is satisfactory.
Example 10: The system of example 9, wherein the implantable medical device is a medical diagnostic device.
Example 11: The system of example 10, wherein the implantable medical device includes a proximal electrode and a distal electrode.
Example 12: The system of any of examples 9 to 11, wherein the implant location includes implant position and implant orientation.
Example 13: The system of any of examples 9 to 12, wherein a spacing of the set of sensors carried by the piercing member corresponds to a spacing of a set of sensors carried by the implantable medical device.
Example 14: The system of any of examples 9 to 13, wherein the metric of the signal indicates one or more of a signal strength or a signal quality.
Example 15: The system of any of examples 9 to 14, wherein the fluid reservoir includes a display configured to display the metric of the signal.
Example 16: The system of any of examples 9 to 15, wherein the fluid includes at least one of anesthetic fluid, saline, or vasoconstrictors.
Example 17: The system of any of examples 9 to 16, wherein the set of sensors include at least one of an electrode array, a photoemitter, a photodetector, or an accelerometer, an inductive sensor, an electromagnetic sensor, or an impedance sensor.
Example 18: The system of any of examples 9 to 17, wherein the fluid reservoir includes a set of input devices, and wherein the processing circuitry is configured to configure the set of sensors based on an input from the one or more input devices.
Example 19: A method includes positioning a set of sensors of a fluid delivery device in subcutaneous tissue of a patient, wherein the fluid delivery device includes: a fluid reservoir configured to eject a fluid into the patient; a piercing member in fluid communication with the fluid reservoir, wherein the piercing member is configured to penetrate the subcutaneous tissue of the patient, and wherein the set of sensors is carried by the piercing member; a plunger configured to eject the fluid from the fluid reservoir through a lumen defined by the piercing member; and communication circuitry; measuring, by the set of sensors, a signal from the subcutaneous tissue; communicating, by the communication circuitry, the signal to an external device; determining, by processing circuitry of the external device, a metric of the signal; determining, by the processing circuitry, whether the metric of the signal satisfies a condition; and based on whether the metric of the signal satisfies the condition, outputting, by the processing circuitry, a notification indicative of whether an implant location for an implantable medical device is satisfactory.
Example 20: The method of example 19, wherein the implant location includes implant position and implant orientation.
Example 21: The method of example 19 or 20, wherein a spacing of the set of sensors carried by the piercing member corresponds to a spacing of a set of sensors carried by the implantable medical device.
Example 22: The method of any of examples 19 to 21, wherein the metric of the signal indicates one or more of a signal strength or a signal quality.
Example 23: The method of any of examples 19 to 22, wherein the fluid reservoir includes a display configured to display the metric of the signal.
Example 24: The method of any of examples 19 to 23, wherein the fluid includes at least one of anesthetic fluid, saline, or vasoconstrictors.
Example 25: The method of any of examples 19 to 24, wherein the set of sensors include at least one of an electrode array, a photoemitter, a photodetector, or an accelerometer, an inductive sensor, an electromagnetic sensor, or an impedance sensor.
Example 26: The method of any of examples 19 to 25, wherein the fluid reservoir includes a set of input devices, and wherein the processing circuitry is configured to configure the set of sensors based on an input from the one or more input devices.

The techniques described in this disclosure may be implemented, at least in part, in hardware, software, firmware, or any combination thereof. For example, various aspects of the techniques may be implemented within one or more microprocessors, digital signal processors (DSPs), application specific integrated circuits (ASICs), field-programmable gate arrays (FPGAs), or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as random access memories (RAM), dynamic random access memories (DRAM), static random access memories (SRAM), magnetic discs, optical discs, flash memories, or forms of electrically programmable memories (EPROM) or electrically erasable and programmable memories (EEPROM). The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

1. A fluid delivery device comprising:
a fluid reservoir configured to contain a fluid;
a piercing member in fluid communication with the fluid reservoir, wherein the piercing member is configured to penetrate subcutaneous tissue of a patient;
a plunger configured to eject the fluid from the fluid reservoir through a lumen defined by the piercing member;
a set of sensors carried by the piercing member, wherein the set of sensors is configured to:
be positioned in the subcutaneous tissue when the piercing member has penetrated the subcutaneous tissue; and
measure a signal from the subcutaneous tissue; and
processing circuitry configured to:
determine a metric of the signal;
determine whether the metric of the signal satisfies a condition; and
based on whether the metric of the signal satisfies the condition, output a notification indicative of whether an implant location for an implantable medical device is satisfactory, wherein the implantable medical device comprises a housing enclosing electronic circuitry.

2. The fluid delivery device of claim 1, wherein the implant location comprises implant position and implant orientation.

3. The fluid delivery device of claim 1, wherein a spacing of the set of sensors carried by the piercing member corresponds to a spacing of a set of sensors carried by the implantable medical device.

4. The fluid delivery device of claim 1, wherein the metric of the signal indicates one or more of a signal strength or a signal quality.

5. The fluid delivery device of claim 1, wherein the fluid reservoir comprises a display configured to display the metric of the signal.

6. The fluid delivery device of claim 1, wherein the fluid comprises at least one of anesthetic fluid, saline, or vasoconstrictors.

7. The fluid delivery device of claim 1, wherein the set of sensors comprise at least one of an electrode array, a photoemitter, a photodetector, an accelerometer, an inductive sensor, an electromagnetic sensor, or an impedance sensor.

8. The fluid delivery device of claim 1, wherein the fluid reservoir comprises a set of input devices, and wherein the processing circuitry is configured to configure the set of sensors based on an input from the one or more input devices.

9. A system comprising:
a fluid delivery device comprising:
a fluid reservoir configured to eject a fluid into a patient;
a piercing member in fluid communication with the fluid reservoir, wherein the piercing member is configured to penetrate subcutaneous tissue of the patient;
a plunger configured to eject the fluid from the fluid reservoir through a lumen defined by the piercing member;
a set of sensors carried by the piercing member, wherein the set of sensors is configured to:
be positioned in the subcutaneous tissue when the piercing member has penetrated the subcutaneous tissue; and
measure a signal from the subcutaneous tissue; and
communication circuitry; and
an external device, wherein the communication circuitry is configured to communicate with the external device, and wherein the external device comprises processing circuitry configured to:
determine a metric of the signal;
determine whether the metric of the signal satisfies a condition; and
based on whether the metric of the signal satisfies the condition, output a notification indicative of whether an implant location for an implantable medical device is satisfactory, wherein the implantable medical device comprises a housing enclosing electronic circuitry.

10. The system of claim 9, wherein the implantable medical device is a medical diagnostic device.

11. The system of claim 10, wherein the implantable medical device comprises a proximal electrode and a distal electrode.

12. The system of claim 9, wherein the implant location comprises implant position and implant orientation.

13. The system of claim 9, wherein a spacing of the set of sensors carried by the piercing member corresponds to a spacing of a set of sensors carried by the implantable medical device.

14. The system of claim 9, wherein the metric of the signal indicates one or more of a signal strength or a signal quality.

15. The system of claim 9, wherein the fluid reservoir comprises a display configured to display the metric of the signal.

16. The system of claim 9, wherein the fluid comprises at least one of anesthetic fluid, saline, or vasoconstrictors.

17. The system of claim 9, wherein the set of sensors comprises at least one of an electrode array, a photoemitter, a photodetector, an accelerometer, an inductive sensor, an electromagnetic sensor, or an impedance sensor.

18. A method comprising:
positioning a set of sensors of a fluid delivery device in subcutaneous tissue of a patient, wherein the fluid delivery device comprises:
a fluid reservoir configured to contain a fluid;
a piercing member in fluid communication with the fluid reservoir, wherein the piercing member is configured to penetrate the subcutaneous tissue of the patient, and wherein the set of sensors is carried by the piercing member;
a plunger configured to eject the fluid from the fluid reservoir through a lumen defined by the piercing member; and
communication circuitry;
measuring, by the set of sensors, a signal from the subcutaneous tissue;
communicating, by the communication circuitry, the signal to an external device;
determining, by processing circuitry of the external device, a metric of the signal;
determining, by the processing circuitry, whether the metric of the signal satisfies a condition; and
based on whether the metric of the signal satisfies the condition, outputting, by the processing circuitry, a notification indicative of whether an implant location for an implantable medical device is satisfactory, wherein the implantable medical device comprises a housing enclosing electronic circuitry.

19. The method of claim 18, wherein the implant location comprises implant position and implant orientation.

20. The fluid delivery device of claim 18, wherein a spacing of the set of sensors carried by the piercing member corresponds to a spacing of a set of sensors carried by the implantable medical device.
